(19) United States
(12) Patent Application Publication
(10) Pub. No.: US 2023/0118038 A1
(43) Pub. Date: Apr. 20, 2023

(54) DETERMINING PERFORMANCE IN FORMULATIONS FOR OIL-CONTAINING PRODUCTS FOR COSMETICS

(71) Applicant: BASF SE, Ludwigshafen (DE)

(72) Inventors: Sandip Bhattacharya, Düsseldorf-Holthausen (DE); Matthias Hloucha, Düsseldorf-Holthausen (DE); Daniela Prinz, Düsseldorf-Holthausen (DE); Wolf Eisfeld, Düsseldorf-Holthausen (DE)

(21) Appl. No.: 17/909,781

(22) PCT Filed: Mar. 12, 2021
(86) PCT No.: PCT/EP2021/056334

§ 371 Date: Sep. 7, 2022

(30) Foreign Application Priority Data
Mar. 13, 2020 (EP) 20163091.0

(51) Int. Cl. G16C 20/30; G16C 20/80; G16C 20/90; G16C 60/00; A61K 8/92; A61Q 19/00

(52) U.S. Cl. CPC G16C 20/30; G16C 20/80; G16C 20/90; G16C 60/00; A61K 8/92; A61Q 19/00; A61K 2800/596

(57) ABSTRACT

A computer implemented method for determining performance properties of an oil-containing product for cosmetics, the oil-containing product for cosmetics comprising different oils forming a mixture, the method comprising the steps of: providing to a processing device via an input channel o performance properties for each of the different oils o a measure for the ratio of the different oils in the mixture, o a data driven model and/or a rigorous model determining with a processing device determined performance properties of the oil-containing product for cosmetics comprising the mixture, based on o the data driven model o the performance properties for each of the different oils o the measure for the ratio of the different oils in the mixture, providing via an output channel o the determined performance properties of the oil-containing product for personal care and/or o the measure for the ratio of the different oils in the mixture and/or o a formulation of the mixture, and or o a formulation of the oil-containing product for personal care.

The present disclosure relates to systems, methods and computer program products for determining performance properties of an oil-containing and/or the surfactant-containing product, in particular for personal care, more particular for cosmetics is proposed, the oil-containing product comprising different oils forming a mixture and/or the surfactant-containing product for cosmetics comprises at least one surfactant and further component forming the mixture.

### BACKGROUND

A clear view of the requirements of the consumer and the identifier of future trends are critical for the development of successful personal care products. With customized products and solutions for cosmetics, the opportunities and possibilities lying within these new trends should be exhausted.

These new trends also include the development of new formulations for oil-containing products for cosmetics. Changes in formulations do have a tremendous influence on the wearing and application comfort of cosmetic products and impart an impression to the consumer regarding the sensory properties of the product.

New formulations for oil-containing products for cosmetics are tested by time consuming and expensive tests.

Sensory tests for example require trained human testers, that apply the new formulation to the skin and then rate on a scale the sensory values for a formulation.

Extensive tests on physico-chemical properties on a new formulation for oil-based products are also required when developing a new formulation.

The object of the present invention relates to a system and a method to determine properties of a personal care product containing at least two oils.

Products in cosmetic industries widely contain mixtures of multiple ingredients including oils. In personal care industry a frequent term for oils is emollients. Oils play an essential part for the moistening effect and are also influencing the resorption of the cosmetic.

Thus, there is a need for a system and a method to predict properties of a cosmetic formulation which contains oil.

US 2002/082745 A1 describes a method and system for selecting and producing a customized cosmetic or pharmaceutical formulation. The system can be implemented in an Internet based system or a stand-alone version, such as a Kiosk. A method of custom formulation is presented which utilizes the user's preferences and profile, as well as external factors. The customized formulation can be directed to a manufacturing facility for on-demand production. Alternatively, a printout of the formulation can be provided for subsequent use at point-of-sale locations, such as a cosmetics store or a pharmacy. The custom formulation software can also be provided in conjunction with a cosmetics manufacturing kit for use in home or business application.

### SUMMARY

According to a first aspect of the invention a computer implemented method for determining performance properties of an oil-containing and/or surfactant-containing product, in particular for personal care, more particular for cosmetics is proposed, the oil-containing product for personal care comprising different oils forming a mixture and/or the surfactant-containing product for cosmetics comprises at least one surfactant and further component forming the mixture, the method comprising the steps of
providing via a communication interface, input composition parameters for each of the different oils to a processing device
providing via the communication interface a data driven model and/or a rigorous model to the processing device
determining with a processing device determined performance properties of the oil-containing and/or the surfactant-containing product, in particular for personal care, more particular for cosmetics comprising the mixture, based on
   the data driven model and
   the composition parameters,
providing via a communication interface
   the determined performance properties of the oil-containing and/or the surfactant-containing product, in particular for personal care, more particular for cosmetics and/or
   the measure for the ratio of the different oils and/or of the surfactant and further component in the mixture and/or
   a formulation of the mixture, and or
   a formulation of the oil-containing and/or the surfactant-containing product, in particular for personal care, more particular for cosmetics.

In general, the data driven model describes the relation between the performance properties of each of the oils in the mixture and the measure for the ratio of the different oils in the mixture and the performance properties of the mixture. The data driven model may be on measurements of performance properties of different oils in mixtures and a measure for the ratio of the different oils in these mixtures. The data driven model describes the relation between the performance properties of each of the surfactants and/or further components in the mixture and the measure for the ratio of the different surfactants and/or further components in the mixture and the performance properties of the mixture. The data driven model may be on measurements of performance properties of surfactants and further components in mixtures and a measure for the ratio of the surfactants and further components in these mixtures. The data driven model may be on measurements of performance properties of different oils and/or surfactants in mixtures and a measure for the ratio of the different oils and/or for the ratio of the surfactants in these mixtures.

The data driven model refers to a model at least partially derived from data. In contrast to a rigorous model that is purely derived using physico-chemical laws. Use of a data driven model can allow describing relations, that cannot be modelled by physico-chemical laws. The use of data driven models can allow to describe relations without solving equations from physico-chemical laws. This can reduce computational power. This can improve speed. The data driven model may be derived from statistics (Statistics 4th edition, David Freedman et al., W. W. Norton & Company Inc., 2004). The data driven model may be derived from machine learning (Machine Learning and Deep Learning frameworks and libraries for large-scale data mining: a survey, Artificial Intelligence Review 52, 77-124 (2019), Springer).

The data driven model may be a regression model. The data driven model may be a mathematical model. The mathematical model may describe the relation between provided performance properties and determined performance properties as a function. The data driven model may be any other machine learning model.

The data driven model may be a machine learning model. The data driven model may be trained based on one or more of “historic” composition parameters, “historic performance properties” or quantum mechanical descriptors such as described in C. C. Pye, T. Ziegler, E. van Lenthe, J. N. Louwen, Can. J. Chem. 87, 790 (2009).

Cosmetics are personal care products. They may include, but are not limited to, products that can be applied to the face, to the body, to the hands/nails, to the feet, to the hair and to the mouth (e.g. skin-care creams, sun screen, lipsticks, deodorants, lotions, powders, perfumes, baby products, bath oils, bubble baths, fingernail and toe nail polish, and hand sanitizer; hair dye, hair sprays, gels shampoo conditioner, bath salts, and body butters) Cosmetics often contain oil or a mixture of different oils. Cosmetics may comprise surfactants and further components. For cosmetics the user experience is highly important. The user experience depends among other things on the performance properties of the different oils in the oil-containing products and/or surfactants and further components in the surfactant-containing products.

Oils in the sense of that application also comprises cosmetic oil components.

Cosmetic oil components of may be oil components selected from the group consisting of fatty acid esters, esters of C6-C28 fatty acids and C6-C28 fatty alcohols, glyceryl esters, fatty acid ester ethoxylates, alkyl ethoxylates, C12-C28 fatty alcohols, C12-C28 fatty acids, Guerbet esters, Guerbet alcohols and Guerbet acids, saturated alkanes, C12-C28 fatty alcohol ethers, vegetable oils, natural essential oils, mineral oil, parafinum liquidum, petrolatum, isoparaffins, preferably from the group consisting of dibutyl adipate (Cetiol® B), phenethyl benzoate, coco-caprylate (Cetiol® C5), coco-caprylate/caprate (Cetiol® LC, Cetiol® C5, Cetiol® C 5C), propylheptyl caprylate (Cetiol® Sensoft), caprylyl caprylate/caprate (Cetiol® RLF), myristyl myristate (Cetiol® MM), capric glycerides, coco-glycerides (Myritol® 331), capryl/caprin-triglyceride (Myritol® 312), capryl/caprin-triglyceride (Myritol® 318), C12-15 alkyl benzoate (Cetiol® AB), PPG-3 benzyl ether myristate, C12-13 alkyl lactate, isodecyl salicylate, alkyl malate, isoamyl laurate, propylheptyl caprylate, butyloctyl salicylate, polycrylene, dicaprylyl carbonate (Cetiol® CC), dicaprylyl ether (Cetiol® OE), 2-octyldodecylmyristate, isohexadecane, dimethyl capramide, squalene, isopropyl isostearate, isostearyl isostearate, decyl oleate (Cetiol® V), oleyl erucate (Cetiol® J 600), cetearyl ethylhexanoate (Luvitol® EHO), octyldodecanol (Eutanol® G), hexyldecanol (Eutanol® G16), volatile linear C8 to C16 alkanes, C10 to C15 alkanes, C11-C13 alkanes (Cetiol® Ultimate), C13-15 alkanes, C15-19 alkanes, C17-23 alkanes, isododecane, undecane, tridecane (Cetiol® Ultimate), dodecane, propylene glycol dipelargonate, diisopropyl sebacate, cetearyl isononanoate (Cetiol® SN), isononyl isononanoate, isocetyl stearoyl stearate, dipentaerithrityl hexacaprylate/hexacaprate, isodecyl neopentanoate, PEG-6 caprylic/capric glycerides (Cetiol® 767), caprylic/capric triglyceride (Myritol® 312, Myritol® 318), ethylhexyl stearate, ethylhexylcocoate, ethylhexyl stearate (Cetiol® 868), dipropylheptyl carbonate (Cetiol® 4 All), hexyl laurate (Cetiol® A), dicaprylyl carbonate, PEG-7 glyceryl cocoate (Cetiol® HE), polyglyceryl-3 diisostearate (Lameform® TGI), lauryl alcohol, methyl canolate (Cetiol® MC), hexyldecyllaurate and hexyldecanol (Cetiol® PGL), hexyldecyl stearate (Eutanol® G 16S), PPG-15 Stearylether (CETIOL® E), ethylhexyl palmitat (CEGESOFT® C24).

Further fatty acid esters are myristyl palmitate, myristyl stearate, myristyl isostearate, myristyl oleate, myristyl behenate, myristyl erucate, myristyl myristate (Cetiol® MM), cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl oleate, isostearyl behenate, isostearyl oleate, oleyl myristate, oleyl palmitate, oleyl stearate, oleyl isostearate, oleyl oleate, oleyl behenate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate,

Also suitable are Guerbet alcohols, Guerbet acids, Guerbet esters, preferably Guerbet esters of linear C6-22 fatty acids with branched alcohols, especially Guerbet esters of linear C6-22 fatty acids with branched alcohols with C6-C18, preferably C8-C10 fatty alcohols more particularly 2-ethyl hexanol, esters of C18-38 alkylhydroxy carboxylic acids with linear or branched C6-22 fatty alcohols, more especially Dioctyl Malate, esters of linear and/or branched fatty acids with polyhydric alcohols (for example propylene glycol, dimer diol or trimer triol) and/or Guerbet alcohols, triglycerides based on C6-10 fatty acids, liquid mono-, di-and triglyceride mixtures based on C6-18 fatty acids, esters of C6-22 fatty alcohols and/or Guerbet alcohols with aromatic carboxylic acids, more particularly benzoic acid, esters of C2-12 dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, Guerbet carbonates based on fatty alcohols containing 6 to 18 and preferably 8 to 10 carbon atoms, esters of benzoic acid with linear and/or branched C6-22 alcohols (for example Finsolv® TN), linear or branched, symmetrical or nonsymmetrical dialkyl ethers containing 6 to 22 carbon atoms per alkyl group such as, for example, dicaprylyl ether (Cetiol® OE), ring opening products of epoxidized fatty acid esters with polyols.

Further suitable oil components are natural products selected from the group of Elaeis guiineensis oil (Cegesoft® GPO), Passiflora incarnata seed oil (Cegesoft® PFO), olive oil, olus oil (Cegesoft® PS6), Butyrospermum parkii butter (Cetiol® SB 45), ethylhexylcocoat (and) Cocos Nucifera Öl (CETIOL® COCO), Shorea stenoptera seed butter (Cegesoft® SH), almond oil, avocado oil, borage oil, canola oil, castor oil, chamomile, coconut oil, corn oil, cottonseed oil, jojoba oil, evening primrose oil, papaya oil, palm oil, hazelnut oil, peanut oil, walnut oil, safflower oil, sesame oil, soybean oil, sunflower oil, sweet almond, a rice bran/wheat germ oil, rosehip oil, Ricinus communis oil, lanolin; hydrogenated vegetable oil, Candelilla cera, Euphorbia vegetable oil (Cegesoft® VP), sterols and derivatives.

In addition, silicones and silicone derivatives such as polydimethylsiloxanes, methicone, dimethicone, cyclomethicone, caprylyl methicone, dimethicone copolyol, undecylcrylene dimethicone, dimethiconol, trimethicone, organo-siloxanes are used as oil components in cosmetic compositions.

Surfactants in the sense of that application may refer to surfactant molecules compatible with both water and oil. The surfactants may be at least one surfactant selected from the group consisting of an amphoteric surfactant, a cationic surfactant, an anionic surfactant, or a nonionic surfactant. For example, the surfactants may be at least one surfactant selected from the group consisting of EO-based nonionic surfactants, sulfates, 1.4 dioxane, isethionates, taurate, sulfates, fatty alcohol ethersulfates, fatty alcohol sulfates, linear dodecyl benzol sulfonates, linear alkyl benzene sulfonates, oleic acid sulfonates, polyalkylene glycols, alcohol ethoxylates, alkyl polyglucosides, amine ethoxylates, aminopolyols, and unsaturated alcohol ethoxylates.

The surfactants may be preferably selected from the group consisting of C8-C16 fatty alcohol glycoside (Plantacare® 818), C8-10 fatty alcohol glucoside (Plantacare® 810), lauryl glucoside (Plantacare® 1200), C8-C16 fatty alcohol glycoside (Plantacare® 2000), cocoamidopropyl betaine (Dehyton® PK 45), sodium laureth sulfate (Texapon® N 70), sodium cocoyl glutamate (Plantapon® ACG 50), sodium lauryl sulfate (Texapon® K 12), ammonium lauryl sulfate (Texapon® ALS 70), sodium laureth sulfate (Texapon® N 701), disodium 2-sulfolaurate (Texapon® SFA), sodium dodecyl sulfate (Texapon® K 14 SP), laureth-7 citrate (Plantapon® LC 7), disodium 2-sulfolaurate (Texapon® SB 3 KC), sodium cocoamphoacetate (and) glycerin (and) lauryl glucoside (and) sodium cocoyl glutamate (and) sodium lauryl glucose carboxylate (Plantapon® SF-N), coco-glucoside (and) disodium lauryl sulfosuccinate (and) glycerin (Plantapon® PSC), sodium lauryl glucose carboxylate (and) lauryl glucoside (Plantapon® LCG Sorb), disodium lauryl sulfosuccinate (Plantapon® SUS), Plantapon Soy, fatty alcohol sulfate (Sulfopon® 1216 G), disodium cocoamphodiacetate (Dehyton® DC), sodium cocoamphoacetate (Dehyton® MC), sodium cocoyl isethionate (Jordapon® SCI), sodium cocoyl isethionate (Jordapon® CI LA) Isethionates, Taurates, and amino acid surfactants.

The further component may be a solution such as a water based and/or oil based solution. For example, a 2% surfactant solution in water may be used.

The measure for the ratio of the different oils in the mixture indicates the relationship in quantity, amount, or size between two or more of the different oils. The measure of the ratio of the different oils may be the relative or absolute. The measure of the ratio of the different oils may be e.g: the weight percentage, the volume percentage, the mixing ratio, molar ratio.

The measure for the ratio of the different surfactants and/or further components in the mixture indicates the relationship in quantity, amount, or size between two or more of the different surfactants and/or further components. The measure of the ratio of the different surfactants and/or further components may be the relative or absolute. The measure of the ratio of the different surfactants and/or further components may be e.g: the weight percentage, the volume percentage, the mixing ratio, molar ratio.

The formulation of the mixture may comprise the identifier of each of the different oils. In particular it may comprise the identifier of each of the different oils constituting the mixture. The formulation of the mixture may comprise the identifier of each of the different surfactants and/or further components. In particular, it may comprise the identifier of each of the different surfactants and/or further components constituting the mixture.

The communication interface provides information from and to the processing device. The communication interface may enable transfer of information with an input device and/or with an output device. The communication interface may enable transfer of information within the processing device. The communication interface may enable transfer of information with a memory of the processing device.

The input device may be a physical and/or a logical input device. The physical input device may be e.g. a keyboard, a mouse, a touchscreen, a touchpad, microphone, a gesture-based control, a database

The output device may be a physical and/or a logical output device. The physical output device may be e.g a display, a monitor.

The logical output device may be e.g. an API, a remote-control function, a software function call, an interface to a database. The output device may be coupled to the communication interface wired or wireless.

The processing device may be e.g a general-purpose computer, a CPU, a microprocessor, a FGPA, a network of computers, a network of CPUs.

Performance properties of an oil or a mixture of different oils may relate to physico-chemical properties of the oil or the different oils, e.g. density, refractive index, surface tension, interfacial tension, spreadability, viscosity, dielectric constant, molecular weight, Equivalent Alkane Carbon Number (EACN).

Performance properties of the surfactants and/or the further components may relate to physico-chemical properties of the surfactants and/or the further components, e.g. dynamic surface tension; dynamic interfacial tension; foam height such as, in particular from t=0 sec to 300 sec, foaming and foam decay, size and count of foam bubbles and their decay; elastic constant of foams (G′), viscosity buildup upon adding salt, sensory feel on hair.

Composition parameters may be composition parameters of a mixture.

In one aspect the composition parameters may comprise a measure for the ratio of the different oils in the mixture and/or a measure for the ratio of the different surfactants and/or further components in the mixture.

In one aspect the composition parameters may comprise performance properties for each of the different oils and/or for each of the different surfactants and/or the further components.

In one aspect the composition parameters may comprise an identifier for each of the different oils and/or for each of the surfactants and/or the further components.

In one aspect the performance properties of each of the different oils are derived from the identifier for each of the different oils and/or the performance properties of each of the different surfactants and/or the further components are derived from the identifier for each of the different surfactants and/or the further components.

In one aspect the performance properties of an oil and/or of a surfactant or a further component may be physico chemical properties.

Physico-chemical properties may be identified by measurements in experiments. Error on the measurement is little (often negligible).

Using physico-chemical properties of an oil or a mixture of oils and/or a surfactant or the further component or a mixture of surfactant and further component has the advantage that they are easily accessible. They may either be measured in a lab or provided from data sheets or databases. A further advantage is that they generally have little error. Generally, the data in the data sheets or databases is derived from measurements.

In one aspect the physico-chemical properties may relate to e.g. density, refractive index, surface tension, interfacial tension, physical spreadability, viscosity, dielectric constant, molecular weight, the equivalent alkane carbon number (EACN).

Refractive Index (optical property): It is a measure of how fast light propagates through oil or a mixture of at least two oils. It is the ratio of speed of light in vacuum divided by speed of light in the product. It is measured using standard device to measure refractive index.

Surface Tension: Force that holds a unit length of interface between oil and air. It is typically measured using “Wilhelmy plate method (plate tensiometer)” method.

Liquid-liquid Interfacial Tension: Force that holds a unit length of interface between oil and water. It is typically measured using “pendant drop” method.

Spreadability/Spreading Value: Area a fixed amount of oil has spread on a collagen surface (substituting human skin) in 10 minutes. This is measured by a method developed in house by Henkel, presently BASF, Düsseldorf.

Viscosity: Flow behavior or rheology of an oil. This may be measured using standard Rheometers.

The dielectric constant may be measured using Qumat 02600 Dekameter device, The EACN method is developed by BASF Dusseldorf [T. H. Förster et al, International Journal of Cosmetic Science, 16, 84-92 (1994)].

The molecular weight may be determined from the chemical compositions.

Liquid-liquid Interfacial tension (IFT): the interfacial tension is measured in [mN/m] versus water and may be measured using the pendant drop method at a temperature of 23+/−2° C. A Dataphysics OCAH 200 high-speed contact angle measuring system having a cannula (DataPhysics Instruments GmbH, Filderstadt, Germany) with a 0.52 mm diameter, is used for the measurement. The cannula is used to form a water droplet in a cuvette filled with the respective oil, and the droplet size is adjusted in 5 μl steps up to the maximum stable volume, in order to ensure the highest sensitivity for the method. For evaluating the droplet size, the LaPlace-Young method is used, and the required densities are determined with an oscillating U-tube density measuring device. The measurement is repeated 10 times and the IFT is given as the mean of these 10 measurements with the standard deviation thereof.

In one aspect the physico-chemical properties may relate to e.g. dynamic surface tension; dynamic interfacial tension; foam height such as, in particular from t=0 sec to 300 sec, foaming and foam decay, size and count of foam bubbles and their decay; elastic constant of foams (G′), viscosity buildup upon adding salt, and sensory feel on hair. All physico-chemical properties are standard physico-chemical properties to characterize surfactants and are known to the skilled person.

In the following, reference is made to oil-containing product representative for both oil-containing products and surfactant-containing products.

In one aspect the performance properties of an oil may be sensory properties of the oil.

Sensory properties mostly determine how an oil-containing product for personal care, in particular cosmetics an oil is perceived on human skin. For making this a measurable property several sensory properties have been defined (e.g. Thickness, Gloss, Powdery Feel, Silicone Feel, etc.).

These may be defined differently in different labs. Sensory properties usually have to be measured by trained panelists and are not available from datasheets/databases. The number of panelists may be about 10 to 100 more particular about 20. It turned out that reliable information can already be derived with about 10 trained panelists.

For each sensory property, the panelists rate the oil-containing product. This rating may be done on a monadic scale (0-100). The final value of a sensory property for rate the oil-containing product is taken as an average or the median value from all panelists.

Since sensory properties are evaluated by humans, albeit trained, its values can have a large scattering (statistical divergence). At the same time, sensory attributes are sought after information as they give valuable information on the customers perception of the oil-containing product for personal care, in particular cosmetics.

Using sensory properties has the advantage that these properties are most relevant for describing the user experience of a cosmetic product. Sensory properties usually have to be measured by trained panelists and are not available from datasheets/databases.

In one aspect the sensory properties relate to e.g. thickness, gloss, powdery feel, silicone feel, wetness, distribution, thickness, rubs to absorbency, oil, grease amount of residue, dryness, gloss, slipperiness, smoothness, thickness of residue, greasy feel %, oily feel %, powdery feel %, silicone feel, slipperiness, and stickiness The individual sensory properties are self-explanatory in accordance to the respective names. These properties may be classified into those measured immediately after application (TI, Time Immediate); in Rub-Out phase (RO) and after N minutes after application. Here N can be but is not limited to 5 minutes, 20 minutes, etc.

The above-mentioned examples are beneficial as they are well suited to describe the user experience of a cosmetic product.

Currently, the performance properties of a new oil-containing product for personal care, in particular cosmetics comprising different oils have to be determined by tests.

For each a measure for the ratio of the different oils in the mixture new tests have to be performed. As lined out above, this is time consuming and costly.

The advantage of the proposed method is that the number of tests required for a new oil-containing product for personal care, in particular cosmetics comprising different oils is greatly reduced or the tests are even eliminated.

The performance properties for each of the different oils may be one performance property for each of the different oils. The one performance property for each of the different oils may be one sensory property. The one performance property for each of the different oils may be one physico-chemical property.

The performance properties for each of the different oils may be a plurality of performance properties for each of the different oils. The performance properties may be any combination of physico-chemical properties. The performance properties may be any combination of sensory properties. The performance properties may be any combination of physico-chemical and sensory properties.

In one aspect the oil-containing product for personal care comprises at least two different oils.

In one aspect the oil-containing product for personal care comprises at least three different oils.

By combining three oils to create a new oil-containing product, a larger variation of performance properties for the new oil-containing product is achieved.

In one aspect the oil-containing product for personal care comprises a mixture ofat least four different oils.

By combining four oils to create a new oil-containing product, an even larger variation of performance properties for the new oil-containing product is achieved.

In one aspect the surfactant-containing product for personal care comprises at least two different surfactants and/or further components. In addition, a plurality of different oils may be comprised such as two, three or four different oils.

In one aspect the surfactant-containing product for personal care comprises at least three different surfactants and/or further components. In addition, a plurality of different oils may be comprised such as two, three or four different oils.

By combining a plurality of oils and/or surfactants and/or further components to create a new oil-containing and/or surfactant-containing product, a larger variation of performance properties for the new oil-containing and/or surfactant-containing product is achieved.

In one aspect the surfactant-containing product for personal care comprises a mixture of at least four different oils and/or surfactants and/or further components.

In an aspect, the step of providing performance properties of each of the oils may be preceded by a step of providing an identifier for each of the oils.

In an aspect the step of providing an identifier for each of the oils may be followed by deriving the performance properties for each of the oils from the identifier for each of the oils.

The identifier for each of the different oils, may be e.g. internal labels, chemical-structure formulas, brand names, CAS number.

Using identifiers of oils rather than performance properties greatly increases usability of the method.

Deriving performance properties for each of the different oils from the identifier may be performed by retrieving the performance properties from a database.

In an aspect, the step of providing performance properties of each of the surfactants and/or further components may be preceded by a step of providing an identifier for each of the surfactants and/or further components. In an aspect the step of providing an identifier for each of the surfactants and/or further components may be followed by deriving the performance properties for each of the surfactants and/or further components from the identifier for each of the surfactants and/or further components.

The identifier for each of the different oils, may be e.g. internal labels, chemical-structure formulas, brand names, CAS number. Using identifiers of oils rather than performance properties greatly increases usability of the method.

Deriving performance properties for each of the different surfactants and/or further components from the identifier may be performed by retrieving the performance properties from a database.

In another aspect a step of providing to the processing device via the communication interface target performance properties of a specific oil or a mixture of oils and/or a specific surfactant or a specific further components and/or a mixture of surfactant and/or further component may be comprised.

In one aspect, the target performance properties may be the performance properties of a known/real oil or oil mixture. This may for example occur, when an undesired oil is sought to be replaced. Oils may be undesired if they are not environmentally friendly. Oils may be undesired if they do not have approval for use in cosmetics.

The target performance properties for a specific oil or for a mixture of oils may be a requirement for a new formulation or a new mixture of oils. This may occur, when a new formulation needs to be designed such that certain performance properties requirements are met. In such cases target performance requirements may be provided by a customer for example based on market research.

In an aspect, the target properties of a specific oil or a specific mixture of oils relates to a mineral oil or a mixture containing at least one mineral/paraffin oil. Throughout this application paraffin oils are mineral oils. Paraffin oils are very common in personal care products due to their valued sensory properties. Despite their advantages in user experience, they are not considered sustainable

This allows to replace the mineral oil or oils with an oil or oils that are more environmentally friendly. In one aspect the environmentally friendly oil(s) constitute natural raw ingredients.

In an aspect, the target performance properties of a specific oil or a specific mixture of oils relates to a silicone-based oil. Silicone-based oils are very common in personal care products due to their valued sensory properties. Despite their advantages in user experience, they are not considered sustainable

This allows to replace the silicone-based oil or oils with an oil or oils that are more environmentally friendly. In one aspect the environmentally friendly oil(s) constitute natural raw ingredients.

In one aspect, the target performance properties may be the performance properties of a known/real surfactant or further component or a mixture of surfactant and further component.

This may for example occur, when an undesired surfactant is sought to be replaced. Surfactant may be undesired if they are not environmentally friendly. Surfactants may be undesired if they do not have approval for use in cosmetics.

The target performance properties for a specific surfactant or further component or for a mixture of surfactants and further component may be a requirement for a new formulation or a new mixture. This may occur, when a new formulation needs to be designed such that certain performance properties requirements are met. In such cases target performance requirements may be provided by a customer for example based on market research or by measurements.

In an aspect, the method step of providing target performance properties of the specific oil or the specific mixture of oils may further comprise the steps
providing an identifier of the specific oil or the specific mixture of oils,
deriving the target performance properties from the identifier of the specific oil or the specific mixture of oils.

The identifier of the specific oil or the mixture of oils, may be e.g. internal labels, chemical-structure formulas, brand names, CAS number,

Using identifiers of oils rather than performance properties greatly increases usability of the method.

In an aspect, the method step of providing target performance properties of the specific surfactant and/or further component or the specific mixture of surfactant and further component may further comprise the steps
providing an identifier of the specific surfactant and/or further component or the specific mixture of surfactant and further component,
deriving the target performance properties from the identifier of the specific surfactant and/or further component or the specific mixture of surfactant and further component.

In an aspect a step comparing by the processing device the target performance properties of a specific oil or a mixture of oils for the oil-containing product, in particular for personal care, more particular for cosmetics with the determined performance properties of the oil-containing product, in particular for personal care, more particular for cosmetics and deriving a result of the comparing step may be comprised.

Comparing by the processing device the target performance properties of a specific oil or a mixture of oils for cosmetics with the determined performance properties of the oil-containing product for cosmetics and deriving a result of the comparing step may allow to easier determine, if a specific oil or specific mixture of oils can be replaced.

The step of comparing may further comprise comparing if the target performance requirements are met.

Meeting the target performance properties may understood as exactly matching or matching within a predefined error margin the target performance properties and the performance properties of the oil-containing product, in particular for personal care, more particular for cosmetics comprising (a mixture of) the different oils. In another aspect the method comprises generating an objective function based on the target performance property and the performance properties of the oil-containing product for personal care comprising a mixture of the different oils. In another aspect the method further comprises the step of minimizing or maximizing the objective function. Meeting the target performance properties may be achieved by the minimum or maximum of the objective function. The objective function may be an error function.

In an aspect a step comparing by the processing device the target performance properties of a specific surfactant and/or further component or the specific mixture of surfactant and further component for the surfactant-containing product, in particular for personal care, more particular for cosmetics with the determined performance properties of the surfactant-containing product, in particular for personal care, more particular for cosmetics and deriving a result of the comparing step may be comprised.

Comparing by the processing device the target performance properties with the determined performance properties of the surfactant-containing product for cosmetics and deriving a result of the comparing step may allow to easier determine, if a specific surfactant or further component or specific mixture of surfactant and further component can be replaced.

The step of comparing may further comprise comparing if the target performance requirements are met.

In another aspect the method comprises generating an objective function based on the target performance property and the performance properties of the surfactant-containing product for personal care comprising a mixture of the different surfactant(s) and/or further component(s). In another aspect the method further comprises the step of minimizing or maximizing the objective function. Meeting the target performance properties may be achieved by the minimum or maximum of the objective function. The objective function may be an error function.

In another aspect the step of providing via the communication interface may further comprise providing the result of the comparison step via the communication interface.

Providing the result of the comparison step may allow to even further easier determine, if a specific oil or specific mixture of oils and/or surfactant or further component and/or mixture thereof can be replaced.

In an aspect a step of varying the composition parameters may be performed.

Varying the composition parameters enables to quickly determine the performance properties of the oil-containing product, in particular for personal care, more particular for cosmetics for a larger variety of oil containing products.

In one aspect the variation step may comprise varying of the measure for the ratio of the different oils.

This allows to quickly determine performance properties based on different mixing ratios.

Varying the measure for the ratio of the different oils can allow to determine the measure for the ratio of the different oils that best meets the target requirements.

In one aspect the variation step may comprise varying changing at least one identifier of the different oils.

By changing at least one identifier of the different oils, at least one oil in the mixture is replaced.

By changing the at least one identifier of the different oils, it is secured that the best combination of oils to meet the target performance can be found.

In one aspect the variation step may comprise adding an additional identifier of the different oils.

This allows to quickly determine performance properties of new oil mixtures, that are based on more than two oils. In case that target performance, requirements cannot be met by an initial mixture, this step enables to add further oils to the mixture, such that the target performance criteria can be met.

The steps of varying the measure for the ratio of the different oils and changing the at least one identifier of the different oils may be implemented independent of each other or implemented together. When the steps of varying the measure of the ratio of the different oils and changing the at least one identifier of the different oils are implemented together, for each combination of identifiers of the different oils the measure for the ratio of the different oils may be varied before the at least one identifier of the different oils is changed.

This provides an efficient way of determining the performance properties that meet the target performance properties.

In an aspect, step of varying the composition parameters may be followed by of providing varied composition parameters as composition parameters. Varied composition parameters are composition parameters that are derived from the varying step.

In another aspect, the step of varying composition parameters may be repeated until the performance requirements are met. In addition, providing varied composition parameters as composition parameters, may also be repeated until the performance requirements are met.

In an aspect the target performance properties may be weighted. The weighting may be determined by the relevance of a certain performance property. For example, in a specific product the viscosity may be more important than the refractive index. By allowing a weighting of the performance properties, requirements from a customer may be best met.

In a second perspective a system, for determining performance properties of an oil-containing and/or surfactant-containing product, in particular for personal care, more particular cosmetics is proposed, the oil-containing product for personal care comprising different oils forming a mixture and/or the surfactant-containing product for cosmetics comprises at least one surfactant and further component forming the mixture,

the system comprising a processing device configured to perform the method steps of the first perspective.

More particular the system may further comprise
a communication interface and
a processing configured for performing the method steps disclosed above.

In an aspect, the system may further comprise a mixing module configured to control mixing oil-containing and/or surfactant-containing product for personal care, in particular cosmetics.

In a third perspective a computer program product for determining performance properties of an oil-containing and/or surfactant-containing product, in particular for personal care, more particular cosmetics is proposed, the oil-containing product for personal care comprising different oils forming a mixture and/or the surfactant-containing product for personal care comprising different surfactant and further component forming the mixture, the computer program product performing the any of the method steps outlined above in relation with the first perspective when run on a processing device.

A computer program may be stored and/or distributed on a suitable medium, such as an optical storage medium or a solid-state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network.

According to a further exemplary embodiment of the present invention, a data carrier or a data storage medium for making a computer program available for downloading is provided, which computer program is arranged to perform a method according to one of the previously described embodiments of the present invention.

It is to be understood that the embodiments described herein are not mutually exclusive of each other, and that one or more of the described embodiments may be combined in various ways, as would be appreciated by one of ordinary skill in the art.

A computer program performing any of the methods of the present invention may be stored on a computer-readable storage medium (e.g., a non-transitory computer-readable storage medium).

A computer-readable storage medium may be a floppy disk, a hard disk, a CD (Compact Disk), a DVD (Digital Versatile Disk), a USB (Universal Serial Bus) drive, a RAM (Random Access Memory), a ROM (Read Only Memory) and an EPROM (Erasable Programmable Read Only Memory). A computer-readable medium may also be a data communication network, for example the Internet, which allows downloading a program code. The methods, systems and devices described herein may be implemented as software in a Digital Signal Processor, DSP, in a micro-controller or in any other side-processor or as hard-ware circuit within an application specific integrated circuit, ASIC, CPLD, FPGA or other suitable device. The present invention can be implemented in digital electronic circuitry, or in computer hardware, firmware, software, or in combinations thereof, e.g. in available hardware of conventional mobile devices or in new hardware dedicated for processing the methods described herein, as will be described in greater detail below.

The disclosure applies to the systems, methods, computer programs, computer readable non-volatile storage media, computer program products disclosed herein alike. Therefore, no differentiation is made between systems, methods, computer programs, computer readable non-volatile storage media or computer program products. All features are disclosed in connection with the systems, methods, computer programs, computer readable non-volatile storage media, catalysts, chemical processes and computer program products disclosed herein.

Certain aspects of the invention are disclosed below in form of numbered embodiments:

1. A computer implemented method for determining performance properties of an oil-containing product for cosmetics, the oil-containing product for cosmetics comprising different oils forming a mixture, the method comprising the steps of:
providing via a communication interface, composition parameters to a processing device
providing via the communication interface a data driven model and/or a rigorous model to the processing device
determining with a processing device determined performance properties of the oil-containing product for cosmetics comprising the mixture, based on
   the data driven model and/or the rigorous model
   and the composition parameters,
providing via an output communication interface
   the determined performance properties of the oil-containing product for cosmetics and/or
   the composition parameters and/or
   a formulation of the mixture, and or
   a formulation of the oil-containing product for cosmetics.

2. The method of clause 1, wherein the composition parameters comprise a measure for the ratio of the different oils in the mixture.

3. The method of clause 1 or 2, wherein the composition parameters comprise performance properties for each of the different oils.

4. The method of any of the preceding clauses, wherein the composition parameters comprise an identifier for each of the oils and the method further comprises a step of deriving performance properties for each of the oils from the identifier.

5. The method of any of the preceding clauses, wherein the performance properties of each of the different oils relate to physico-chemical properties of each of the different oils.

6. The method of any of the preceding clauses, wherein the performance properties of each of the different oils relate to sensory properties of each of the of the different oils.

7. Method according to any of the preceding clauses, wherein oil-containing product for cosmetics comprises at least two different oils, at least three different oils or at least four different oils.

8. The method of any of the preceding clauses further comprising the step of providing to the processing device via the communication interface target performance properties of a specific oil or a mixture of oils for cosmetics.

9. The method of clause 8, wherein the method step of providing target performance properties is preceded by the steps
providing an identifier of a specific oil or a specific mixture of oils,
deriving the target performance of the specific oil or the specific mixture of oils properties from the identifier of the specific oil or the specific mixture of oils.

10. The method according to clauses 8 or 9, wherein the method further comprises the step of comparing by the processing device the target performance properties of a specific oil or a mixture of oils for cosmetics with the determined performance properties of the oil-containing product for cosmetics and deriving a result of the comparing step.

11. The method of clause 10, wherein the step of comparing comprises comparing if the target performance requirements are met.

12. The method according to clause 10 or 11, wherein providing via the output channel further comprises providing the result of the comparing step.

13. The method according to any of clauses 10 to 12, further comprising the step of varying the composition parameters.

14. The method of clause 13, wherein the step of varying the composition parameter comprises varying the measure for the ratio of the different oils.

15. The method of any of clauses 13 or 14, wherein step of varying the composition parameter comprises changing at least one identifier of the different oils.

16. The method of any of clauses 13 to 15, wherein the step of varying the composition parameters comprises adding an additional identifier of an oil.

17. The method according to any of clauses 13 to 16, further comprising the step of providing the varied composition parameters as composition parameters.

18. The method of clause 17, further comprising the step of repeating the method of clause 17, until the target performance properties are met.

19. The method of any of clauses 8-18, wherein the target performance properties of a specific oil or a mixture of oils for cosmetics relate to a silicone-based oil.

20. The method of any of clauses 8-18 wherein, the target performance properties of a specific oil or a mixture of oils for cosmetics relate to a mineral/paraffin oil.

21. The method of any of clauses 8-18 wherein, the target performance properties of a specific oil or a mixture of oils for cosmetics relate to any other real or fictive oil.

22. A system for determining performance properties of an oil-containing product for cosmetics, the oil-containing product for cosmetics comprising different oils forming a mixture, the system comprising:
a communication interface, and
a processing device configured to perform the method according to any of clauses 1 to 21

23. Computer program product that, when run on a processing device performs the method according to any of clauses 1 to 21.

24. A computer implemented method for determining performance properties of an oil-containing and/or surfactant-containing product for cosmetics, the oil-containing product for cosmetics comprising different oils forming a mixture and/or the surfactant-containing product for cosmetics comprises at least one surfactant and further component forming the mixture, the method comprising the steps of:
providing via a communication interface, composition parameters to a processing device
providing via the communication interface a data driven model and/or a rigorous model to the processing device
determining with a processing device determined performance properties of the oil-containing and/or the surfactant-containing product for cosmetics comprising the mixture, based on
   the data driven model and/or the rigorous model
   and the composition parameters,
providing via an output communication interface
   the determined performance properties of the oil-containing and/or the surfactant-containing product for cosmetics and/or
   the composition parameters and/or
   a formulation of the mixture, and or
   a formulation of the oil-containing and/or the surfactant-containing product for cosmetics.

25. The method of clause 24, wherein the composition parameters comprise a measure for the ratio of the different oils and/or surfactant and the further component in the mixture.

26. The method of clause 24 or 25, wherein the composition parameters comprise performance properties for each of the different oils.

27. The method of any of the clauses 24 to 26, wherein the composition parameters comprise an identifier for each of the oils and/or surfactant and the further component and the method further comprises a step of deriving performance properties for each of the oils and/or surfactant and the further component from the identifier.

28. The method of any of the clauses 24 to 27, wherein the performance properties of each of the different oils and/or surfactant and the further component relate to physico-chemical properties of each of the different oils and/or surfactant and the further component.

29. The method of any of the clauses 24 to 28, wherein the performance properties of each of the different oils and/or surfactant and the further component relate to sensory properties of each of the different oils and/or surfactant and the further component.

30. The method according to any of the clauses 24 to 29, wherein oil-containing product for cosmetics comprises at least two different oils, at least three different oils or at least four different oils.

31. The method of any of the preceding clauses 24 to 30, further comprising the step of providing to the processing device via the communication interface target performance properties of a specific oil or a mixture of oils and/or surfactant and the further component or a mixture of surfactant and the further component for cosmetics.

32. The method of clause 31, wherein the method step of providing target performance properties is preceded by the steps
providing an identifier of a specific oil or a specific mixture of oils and/or a specific surfactant or a specific further component or a specific mixture of surfactant and the further component,
deriving the target performance of the specific oil or the specific mixture of oils properties and/or of the specific surfactant or the specific further component or the specific mixture of surfactant and the further component from the identifier of the specific oil or the specific mixture of oils and/or of the specific surfactant or the specific further component or the specific mixture of surfactant and the further component.

33. The method according to clause 31 or 32, wherein the method further comprises the step of comparing by the processing device the target performance properties of a specific oil or a mixture of oils and/or a specific surfactant or a specific further component or a specific mixture of surfactant and the further component for cosmetics with the determined performance properties of the oil-containing and/or the surfactant-containing product for cosmetics and deriving a result of the comparing step, wherein the step of comparing comprises comparing if the target performance requirements are met, wherein providing via the output channel further comprises providing the result of the comparing step.

34. The method of clause 33, further comprising the step of varying the composition parameters, further comprising the step of providing the varied composition parameters as composition parameters.

35. The method of clause 34, further comprising the step of repeating the method of clause 34, until the target performance properties are met.

36. The method of clauses 34 or 35, wherein the step of varying the composition parameter comprises varying the measure for the ratio of the different oils and/or the surfactant or the further component and/or, wherein step of varying the composition parameter comprises changing at least one identifier of the different oils and/or the surfactant or the further component and or comprises adding an additional identifier of an oil and/or of a surfactant or of a further component.

37. The method of any of clauses 31-36, wherein the target performance properties of a specific oil or a mixture of oils for cosmetics relate to a silicone-based oil or a mineral/paraffin oil.

38. The method of any of clauses 31-36, wherein the target performance properties of a specific surfactant or a specific further component or a mixture of surfactant and further component relate to alkoxylate and sulfate containing surfactants.

40. A system for determining performance properties of an oil-containing and/or surfactant-containing product for cosmetics, the oil-containing product for cosmetics comprising different oils forming a mixture and/or the surfactant-containing product for cosmetics comprises at least one surfactant and further component forming the mixture, the system comprising:
a communication interface, and
a processing device configured to perform the method according to any of clauses 24 to 38

41. Computer program product that, when run on a processing device performs the method according to any of clauses 24 to 38.

42. Use of a system for determining performance properties of an oil-containing and/or a surfactant-containing product for cosmetics according to clause 40 for production of an oil containing product and/or of a surfactant-containing product for cosmetics.

### BRIEF DESCRIPTION OF THE DRAWINGS

As example, embodiments of the present invention are illustrated in the appended drawings. It is to be noted, however, that the appended drawings illustrate only particular aspects of the present invention and are therefore not to be considered limiting of its scope. The present invention may encompass other equally effective embodiments.

FIG. 1 illustrates an example method/flow-chart for determining the performance properties of an oil-containing product.

FIG. 2 shows an example of a conceptual device for determining performance property of an oil-containing product.

FIG. 3 illustrates how an example performance property, Surface Tension (SFT), for a binary oil mixture, varies with mixing ratio. SFT for the mixture is predicted using a data-driven model.

FIG. 4: illustrates that for several oil mixtures and for four different performance properties, the estimates obtained by the corresponding data driven (linear) models agrees excellently with their corresponding experimental values.

FIG. 5: a) depicts the sub-linear, behavior of a binary mixture of oils in terms of its viscosity. b) illustrates that a data driven model (log-log) is able to predict accurately the viscosities of several oil mixtures.

FIG. 6: Illustrates the detailed workflow of the present invention giving the performance of the optimized oil mixture, names of the mixture constituents, and mixing ratio that matches best to a target oil or an oil mixture or any performance profile.

FIG. 7: Shows the comparison between the performance profiles of Cyclomethicone and an optimized oil mixture derived from the method outlined in this invention.

FIG. 8: Shows an embodiment of a system or determining performance properties of an oil-containing and/or surfactant-containing product for cosmetics.

### DETAILED DESCRIPTION

The present disclosure provides a method for determining performance properties of an oil-containing product for cosmetics, the oil-containing product for cosmetics comprising different oils forming a mixture.

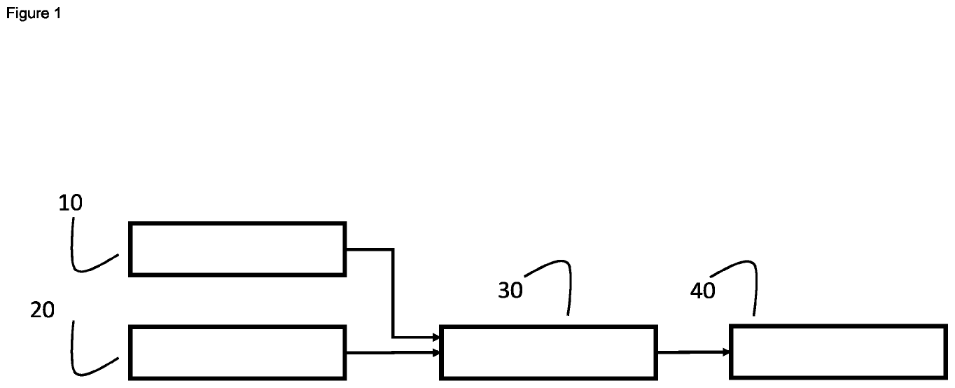

FIG. 1 shows an example the disclosed method in form of a simplified flow chart. In this example determining performance properties of a mixture of two different oils is described. The first of the different oils is dicaprylyl ether (Cetiol® OE) The second of the different oils is coco-caprylate/caprate (Cetiol® LC).

At step 100 composition parameters are provided via a communication interface to a processing device. In this example the composition parameters comprise an identifier for each of the different oils and a step of deriving performance properties for each of the oils from the identifier is performed. In cases where the performance parameters are derived from the identifier, the derived performance parameters are also provided as composition parameters. The identifier in this example are the brand names of each of the oils. In this example the brand names of are entered via a keyboard.

Other means for providing the identifier of each of the oils are also possible, for example providing the identifier of each of the oils from a database.

In this embodiment the performance properties for each of the oils are derived from a database.

Additionally, or alternatively, the method may comprise a step of selecting one or more of the performance properties of each of the different oils. This allows reducing the number of performance properties of each of the oils to the performance properties that are relevant for the task. This reduces computational power.

In this example, the performance properties for each oil is a single physico-chemical performance property. More specifically, the performance properties for each oil is the surface tension (SFT), see table below.

TABLE 1

| | | | | | mixture | Mixture |
|---|---|---|---|---|---|---|
| | | | | | perfor- | perfor- |
| Perfor- | Oil₁ | Oil₂ | | | mance | mance |
| mance | (Cetiol | (Cetiol | x₁ | x₂ | property | property |
| property | OE) | LC) | [%] | [%] | (determined) | (measured) |
| SFT | 27.26 | 29.80 | 25 | 75 | 29.16 | 29.00 |
| (mN/m) | | | | | | |

The composition parameters in this example further comprise, a measure for the ratio of the different oils. In this is example, the measure for the mixing ratio of the first of the different oils is entered by a keyboard.

In other examples the measure for the mixing ratio of the oils may be derived from an identifier of a mixture of oils containing these oils.

In this example the measure for the mixing ratio of the second oil is derived from the measure for the mixing ratio of the first of the different oils. In other cases, the measure for mixing ratio for each of the different oils may be provided for each oil separately.

In this example at step 200 a data driven model is provided via the communication interface to the processing device. In this example, the data driven model is a linear mixing model. On other examples data driven model may be a log-log model. In other examples other data driven models e.g. as mentioned above may be provided.

At step 300 with a processing device determined performance properties of the oil-containing product for cosmetics comprising the mixture are determined with the processing device, based on the data driven model and/or the rigorous model and the composition parameters.

The linear model in this example can be described by SFT(mixture₂)=Σ₁²SFT(oilᵢ)·x₁. The data driven model in this example was derived by linear regression on measurements of different ratios. Index 2 indicates that the mixture comprises two different oils. The term xᵢ. relates to the relative portion of oil; in the mixture in percent.

When the performance properties comprise more than one performance property for each of the different oils the performance of each oil may be provided as a vector \vec{prop}(oilᵢ) for oil i. For the example given in table 2, the properties are SFT, RI, spreading value and density. And the mixing ratio x₁ is 25% and x₂=100−25=75%.

TABLE 2

| | Oil₁ | Oil₂ | | Performance | Performance |
|---|---|---|---|---|---|
| Performance | (Cetiol | (Cetiol | | properties | properties |
| property | OE) | LC) | x₁ | determined | measured |
| SFT (mN/m) | 27.26 | 29.80 | 25 | 29.16 | |
| RI | 1.433 | 1.445 | 25 | 1.442 | 1.442 |
| Spreading value | 1607 | 557 | 25 | 819 | 729 |
| (mm²/10 min) | | | | | |
| Density (gm/cm³) | 0.805 | 0.856 | 25 | 0.8432 | 0.8427 |

The performance property vector for the example in table 2 would therefore look like

$${prop}^{⟶} = ( \begin{matrix}SFT \\ RI \\ spreading value \\ density\end{matrix} )$$

For a mixture of two oils the step of determination of the determined properties of the mixture could be described in a general way as:

\vec{prop}(mixtureᵢⱼ)=ƒ{\vec{prop}(oilᵢ),\vec{prop}(oilⱼ),xᵢⱼ}

with xᵢⱼ: mixing ratio of the two different oils and \vec{prop}(oilᵢ): the vector with performance properties of oil/and \vec{prop}(mixtureᵢⱼ): the vector with performance properties of the oil mixture. The function f is the mathematical description of the data driven model.

An example of a linear data driven model for a multitude of n oils is described by the formula below.

$${prop}^{⟶} ( {mixture}_{ij… n} ) = {\sum }_{i}^{n} {prop}^{⟶} ( {oil}_{i} ) \cdot {x}_{i}$$

At step 400 providing via a communication interface, the determined performance properties of the oil-containing product for cosmetics is performed. In this example the performance properties of the oil-containing product for cosmetics are provided to a display.

Providing to the display has the advantage that the information is readily available for a user.

In other examples the communication interface may provide the determined performance properties of the oil-containing product for cosmetics to a database. Providing to a database has the advantage, that the information can later be retrieved and may be available for later use.

In a further example, the communication interface may provide the determined performance properties of the oil-containing product for cosmetics to a database and a display simultaneously.

Providing to the database and the display simultaneously has the advantage, that the information can later be retrieved and may be available for later use and is also readily available to the user.

The method may within the scope of the invention be extended to three, four, or more than four different oils.

In an alternative, the performance property may be any of the physico-chemical properties, or any of the sensory properties. In a further alternative the performance property may comprise more than one performance property for each of the different oils.

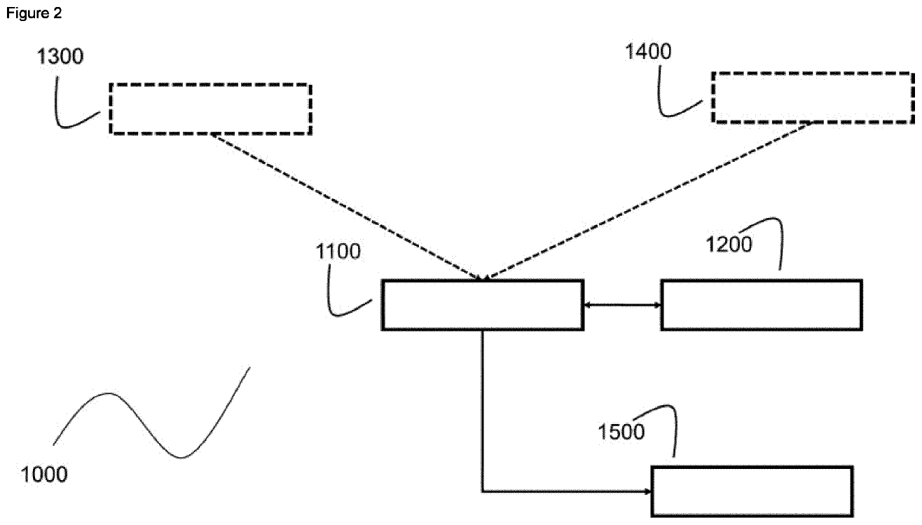

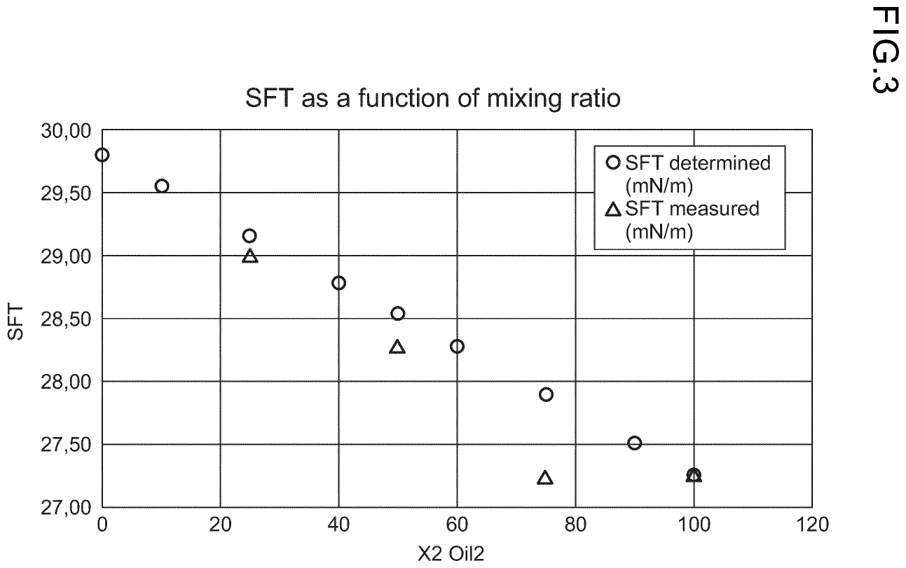

In FIG. 2 an example of a system 1000 for determining the performance properties of an oil-containing product, in particular for personal care, more particular for cosmetics is shown.

The system is configured for performing the method steps 100 to 400 described above in context with FIG. 1. FIG. 2 focuses on illustrating the conceptual device in this invention.

In this embodiment the system comprises a communication interface 1100 for providing composition parameters to a processing device 1200, and for providing a data driven model and/or a rigorous model to the processing device.

The system further comprises a physical input device 1300 connected to the communication channel. In this example the physical input device is a keyboard.

In other embodiments the performance properties for each of the different oils may be provided by the physical input device. This allows the use of the system for determination of performance properties of the oil-containing product for cosmetics even when the performance properties for each of the different oils are not available for all of the different oils. This can increase flexibility of the system.

The system further comprises a logical input device 1400. The logical input device in this case is a database in which performance properties for different oils and/or mixtures of oil are stored.

In this example, the measure for the ratio of the different oils in the mixture is provided as a composition parameter to the communication interface 1100 via database 1400.

In other examples the measure for the ratio of the different oils in the mixture may be provided as composition parameters to the communication interface by entering into keyboard 1300.

In this example, the data driven model and/or a rigorous model is provided to the communication interface 1100 via database 1400.

The system further comprises an output device 1500. The communication interface provides the determined performance properties of the oil-containing product for personal care and/or the measure for the ratio of the different oils in the mixture and/or a formulation of the mixture, and or a formulation of the oil-containing product for cosmetics to the output device. In this example the output device is a display. A formulation of the oil containing product may contain additives such as e.g. water, preservatives, emulsifiers.

In a further example the output device may be a database. In yet another example, the output device may be a combination of a display and a database.

The processing device 1200 is configured to perform the steps outlined in context with the method described in FIG. 1.

FIG. 3 shows a plot of the determined performance properties of an oil-containing product, in particular for personal care, more particular for cosmetics, the oil-containing product for personal care comprising different oils forming a mixture, for various measures for the ratio of the different oils in the mixture. The X-axis refers to the ratio of oil, in the mixture. Oil in this example is Cetiol OE, the second oil in this example is Cetiol LC. The determined performance property is SFT. It can be seen, that there is a is a linear relationship between the measure for the ratio of the different oils in the mixture and the determined performance property. Some measured performance properties are also shown provided at selected mixing ratios. Examples of determined and measured values for different mixing ratios are shown in table 3.

TABLE 3

| x₁ Oil₁ | x₂ Oil₂ | SFT determined | SFT measured |
|---|---|---|---|
| (Cetiol OE) in % | (Cetiol LC) | (mN/m) | (mN/m) |
| 100 | 0 | 27.26 | 27.26 |
| 90 | 10 | 27.514 | |
| 75 | 25 | 27.90 | 27.23 |
| 60 | 40 | 28.276 | |
| 50 | 50 | 28.53 | 28.28 |
| 40 | 60 | 28.784 | |
| 25 | 75 | 29.16 | 29.00 |
| 10 | 90 | 29.546 | |
| 0 | 100 | 29.8 | |

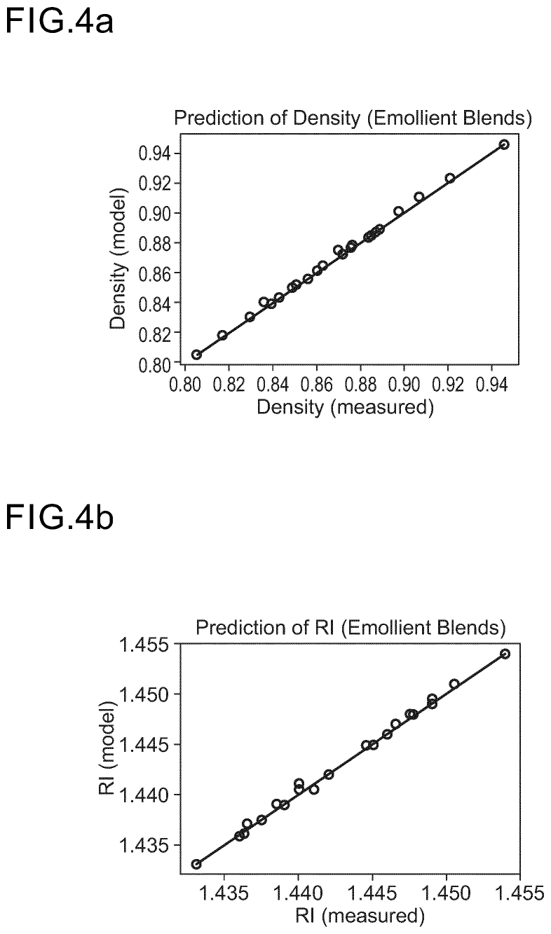

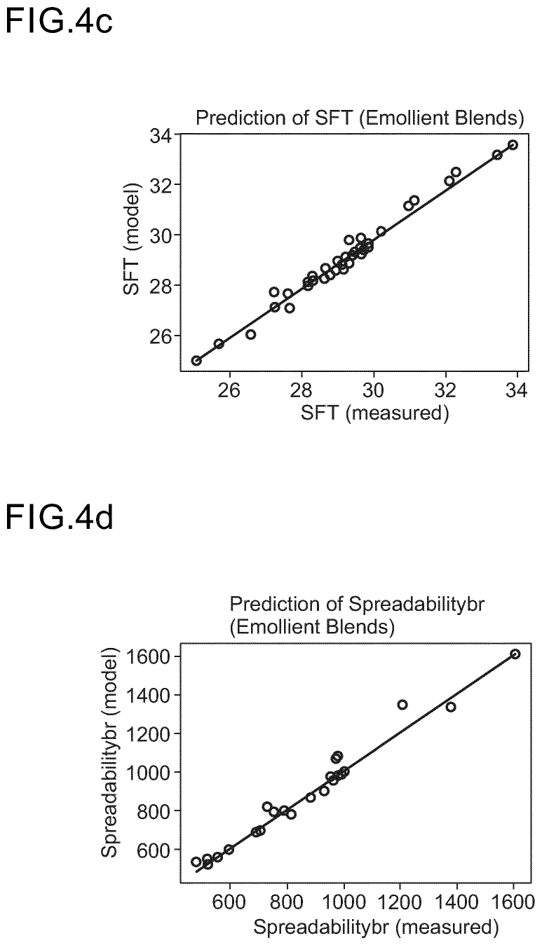

FIGS. 4 a-d show examples of physico-chemical properties where the determined performance property can be determined based on a data driven model. In these cases, the data driven model is a simple linear model. Each data point relates to a ratio of different oil mixtures with varying chemistries. The data driven model accurately captures the performance for several mixtures.

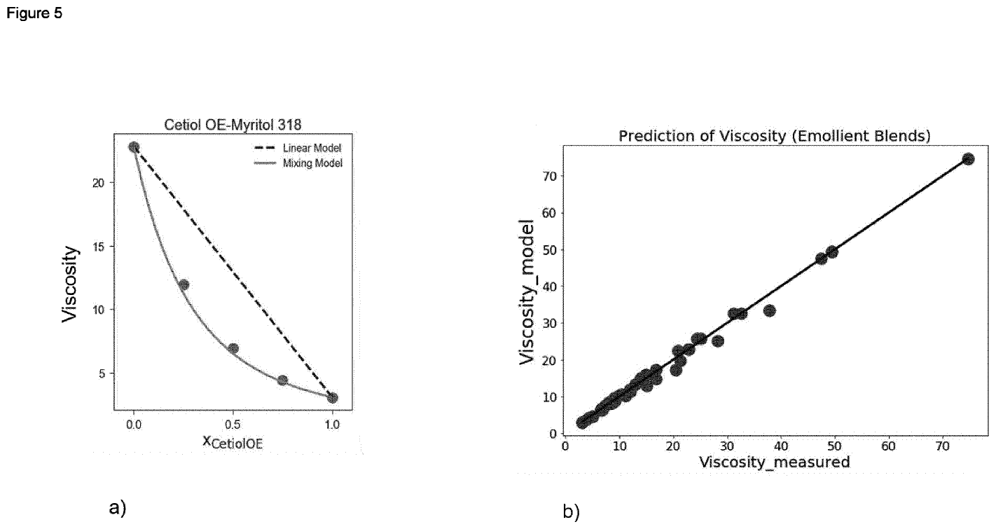

FIGS. 5 a)-b) show that non-linear data driven models may also occur. In this example the viscosity of dicaprylyl ether (Cetiol® OE) and caprylic/capric triglyceride (Myritol® 318) have been provided as the performance property. FIG. 5 a) shows the plot of a measured viscosity of the mixture of dicaprylyl ether (Cetiol® OE) and caprylic/capric triglyceride (Myritol® 318) at various ratios. It can be clearly seen that the viscosity of the mixture does not change linear with the ratio between the two oils, but rather follows a sublinear log-log behavior. Also shown is a fit to the derived data-driven model, which is a log-log model. FIG. 5b) shows the behavior of the log-log model in comparison to the experimentally measured viscosity of several oil mixtures with different chemistries. The model fitting viscosities of oil mixture is of the following form:

loglog[\vec{prop}(mix_{ij . . . n})]=loglog[\vec{prop}(oilᵢ)]·xᵢ

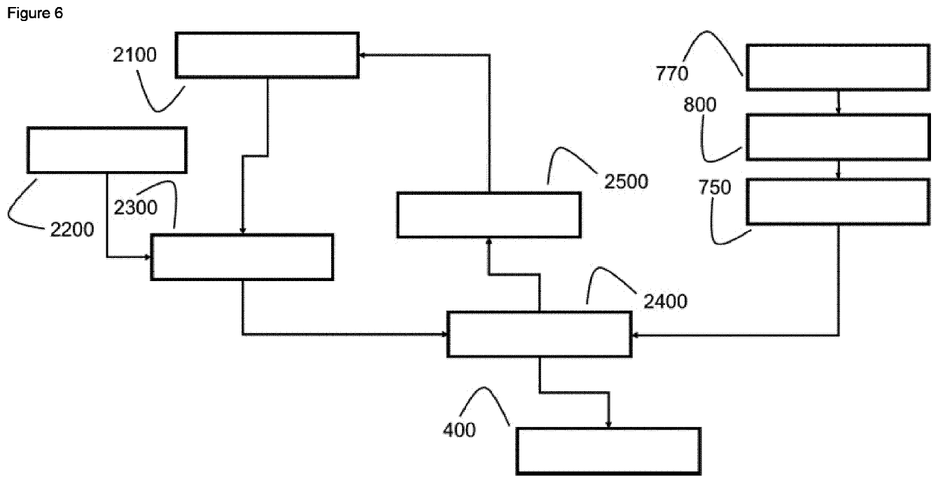

FIG. 6 shows a flowchart of a further aspect. In this example an alternative use of the method for determining performance properties of an oil-containing product for cosmetics is disclosed the oil-containing product for cosmetics comprising different oils forming a mixture.

At step 750 target performance properties of an oil or a mixture of oils for cosmetic are provided to the processing device via the communication channel. In this example the step of providing to the processing device via the communication channel target performance properties of an oil or a mixture of oils for cosmetics is preceded by the step 770 of providing an identifier of the specific oil or the specific mixture of oils, and step 800 deriving target performance properties from the identifier of the specific oil or the specific mixture of oils. In this example the method is applied to only one specific oil. The identifier of the specific oil in this example is cyclopentasiloxane/cyclomethicone. The task which this flowchart is describing is to identify new oil mixtures and their ratios that matches the properties of the specific/target oil, cyclomethicone.

The target performance properties used in this example include: viscosity, spreadability, density, RI, IFT, SFT as physical properties and include: thickness, gloss, powdery feel, silicone feel, wetness, distribution, thickness, rubs to absorbency, oil, grease amount of residue, dryness, gloss, slipperiness, smoothness, thickness of residue, greasy feel %, oily feel %, powdery feel %, silicone feel, slipperiness, and stickiness as sensory properties. In other examples the target performance properties may only be one physical property or one sensory property or any combination of physical and/or sensory properties. The target performance properties are derived from the database.

In this example at step 2200 a data driven model is provided via a communication interface to a processing device. In this example, providing a data driven model comprises providing a data driven model for each performance property.

At step 2100 composition parameters are provided via the communication interface to the processing device. In this example the composition parameters comprise an identifier for each of the different oils and a step of deriving performance properties for each of the oils from the identifier is performed.

In cases where the performance parameters are derived from the identifier, the derived performance parameters are also provided as composition parameters. The identifier in this example are the brand names of each of the oils. In this example the brand names were selected from a list of oils in a database. Other means of providing the identifiers are also possible, for example providing the identifier of each of the oils via a keyboard.

Providing the identifiers via a database is in particular useful, if the method is automized.

The identifiers in this example were based on a binary system of oils. In other examples tertiary and quarterly systems are also possible. In principle, there is no upper limit for the number of different oils. The binary system of oils in this example comprises Cetiol C5 and Cetiol Ultimate, both of which are trademarks of BASF.

In this example the performance parameters for each of the different oils are derived from the identifier. In this embodiment the performance properties for each of the oils are derived from a database. The derived performance parameters are also provided as composition parameters.

Consequently, the performance properties of Cetiol C5 and Cetiol Ultimate are also provided as composition parameters. In this example, the performance properties that are provided comprise viscosity, spreadability, density, RI, IFT, SFT as physical properties and include: thickness, gloss, powdery feel, silicone feel, wetness, distribution, thickness, rubs to absorbency, oil, grease amount of residue, dryness, gloss, slipperiness, smoothness, thickness of residue, greasy feel %, oily feel %, powdery feel %, silicone feel, slipperiness, and stickiness as sensory properties. In this case the provided performance properties are identical to the target performance properties. This allows to best meet the target performance properties. In other examples only a subset of target performance properties may be provided as performance properties of each of the oils.

Additionally, or alternatively, the method may comprise a step of selecting one or more of the performance properties of each of the oils. This allows reducing the number of performance properties of each of the oils to the performance properties that are relevant for the task. This reduces computational power.

In this example the composition parameters also comprise a measure for the ratio of the different oils in the mixture. In this is example, the mixing ratio is fixed and reflects an initialization value.

In other examples, the measure for the mixing ratio of the first of the different oils may be entered by a keyboard and the measure for the mixing ratio of the second oil is derived from the measure for the mixing ratio of the first of the different oils.

In other cases, the measure for mixing ratio for each of the different oils may be provided for each of the oils separately.

In this example, the measure for the mixing ratio of the first of the different oils is x₁=1.

At step 2300 the performance properties of the oil containing product for cosmetics comprising the mixture are determined with the processing device, determining with a processing device determined performance properties of the oil-containing product for cosmetics, based on the data driven model, the performance properties for each of the different oils, the measure for the ratio of the different oils in the mixture.

At step 2400 the target performance properties of a specific oil or a mixture of oils for cosmetics are compared with the determined performance properties of the oil-containing product for cosmetics. A result of the comparing step is generated.

In this example, comparing the target performance properties of a specific oil or a mixture of oils for cosmetics with the determined performance properties of the oil-containing product for cosmetics includes calculating a value of an error function, according to

$$erf ( ij , {oil}_{R} ) = {\sum }_{prop} {\vert {prop}^{⟶} ( {oil}_{R} ) - {prop}^{⟶} ( {oil}_{{mixture}_{ij}} ) \vert}^{2} ,$$

wherein i, j are indices related to different oils, \vec{prop}(oil_{R}) relates to the target properties of the selected oil or mixture of oils and \vec{prop}(oilₘᵢₓₜᵤᵣₑ_{ᵢⱼ}) relates to the determined performance properties of the mixture of oils. The result of the comparing step in this example is the value of the error function. In other cases, the result may be a binary value reflecting, whether the target performance properties are matched.

At step 2500 the composition parameters of the mixture of oils are varied. This composition parameter may be the measure for the ratio of the different oils in the mixture. In this example, the step of varying the composition parameter comprises varying the measure for the ratio of the different oils. In this example the varying step is repeated until the performance requirements are met. In this example meeting the performance requirements relates to minimizing the error function described above.

The result of the comparison step may be provided via the communication interface. This may result in providing a table shown below.

| Oilᵢ | Oilⱼ | Xₒₚₜ (oilᵢ ratio) | Erf * |
|---|---|---|---|
| Cetiol C5 | Cetiol Ultimate | 0.75 | 0.58 |

In this example, the step varying the composition parameters further comprises changing at least one identifier of the different oils.

In this example, the at least one identifier of the different oils that is changed is the brand name of Cetiol Ultimate. In this example the identifier is changed to Cetiol RLF.

The varied composition parameters are then provided as composition parameters.

By this an iterative optimization process is enabled.

Finally, in the table below, for the target silicone oil, Cyclomethicone, the corresponding mixing ratios for four example binary mixtures and the values of the optimized error function computed through the routine outlined in FIG. 6 are shown. This table was derived by iteratively changing the identifier of oilⱼ, and providing a result of the comparison step together with the minimum of the error function.

| Oilᵢ | Oilⱼ | Xₒₚₜ (oilᵢ ratio) | Erf * |
|---|---|---|---|
| Cetiol C5 | Cetiol Ultimate | 0.75 | 0.58 |
| Cetiol C5 | Cetiol RLF | 0.45 | 0.66 |
| Cetiol C5 | Cetiol A | 0.50 | 0.71 |
| Cetiol C5 | Cetiol Sensoft | 0.15 | 0.71 |

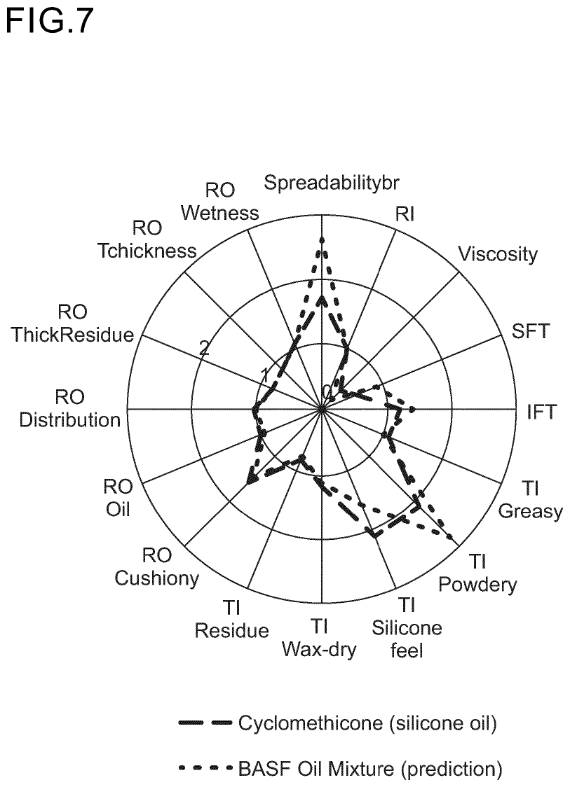

FIG. 7 illustrates an example match between the performance profile of a mixture \vec{prop}(mixtureᵢⱼ) and that of a example target silicone-oil, \vec{prop}(Cyclomethicone), using the method outlined in FIG. 6.

At step 500, the final determined performance properties of the oil-containing product for personal care may be provided via the communication interface.

In this example, along with the performance properties of the oil-containing product for personal care, the constituent oils (names) and mixing ratio are provided to a display.

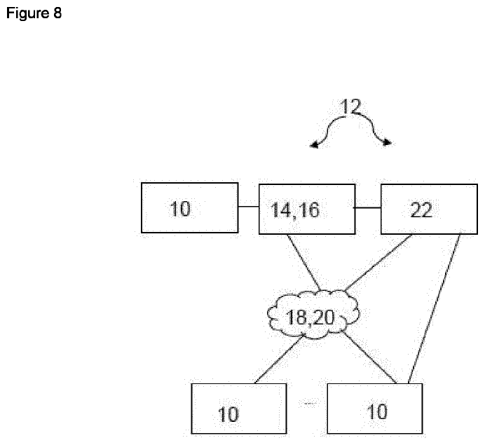

FIG. 8 shows an embodiment of a system 12, in particular an internet-based system 12, for determining performance properties of an oil-containing and/or surfactant-containing product for cosmetics. The Internet-based system 12 may comprise a server 14, e.g. comprising a processing device 16, which can be accessed via the communication interface 18 such as a network 20, such as the Internet, by one or more client devices 10. The client devices 10 may be computer terminals accessible by a user and may be customized devices, such as data entry kiosks, or general purpose devices, such as a personal computer. Preferably, the server 14 is an HTTP server and is accessed via conventional Internet web-based technology. The server 14 may be connected to a further client device 10 and, either directly or indirectly through the network, to a manufacturing facility 22. The server 14 may be configured to trigger a request of initiating the method for determining performance properties of an oil-containing and/or surfactant-containing product for cosmetics. The manufacturing facility 22 can be located proximate to the server 14 and be part of an overall customized ordering and manufacturing system. Alternatively, manufacturing facility 22 may be remotely located from both the server 14 and the client devices 10. For example, the performance properties of an oil-containing and/or surfactant-containing product for cosmetics can be forwarded directly, such as via e-mail, to the manufacturing facility 22. In yet a further embodiment the manufacturing facility 22 can be located proximate a client device 10. This arrangement is particularly well suited for a kiosk-based on-demand manufacturing system, e.g., such as may be located in a point-of-sale establishment. These three potential connections to the manufacturing facility 22 are illustrated in FIG. 8. Multiple manufacturing facilities 22 located at different places may be provided or only one connection may be implemented.

1. A computer implemented method for determining performance properties of an oil-containing and/or surfactant-containing product for cosmetics, the oil-containing product for cosmetics comprising different oils forming a mixture and/or the surfactant-containing product for cosmetics comprises at least one surfactant and further component forming the mixture, the method comprising:
providing via a communication interface, composition parameters to a processing device
providing via the communication interface a data driven model and/or a rigorous model to the processing device
determining with a processing device determined performance properties of the oil-containing and/or the surfactant-containing product for cosmetics comprising the mixture, based on
the data driven model and/or the rigorous model
and the composition parameters,
providing via an output communication interface
the determined performance properties of the oil-containing and/or the surfactant-containing product for cosmetics and/or
the composition parameters and/or
a formulation of the mixture, and or
a formulation of the oil-containing and/or the surfactant-containing product for cosmetics.

2. The method of claim 1, wherein the composition parameters comprise a measure for the ratio of the different oils and/or surfactant and the further component in the mixture.

3. The method of claim 1, wherein the composition parameters comprise performance properties for each of the different oils.

4. The method of claim 1, wherein the composition parameters comprise an identifier for each of the oils and/or surfactant and the further component and the method further comprises a step of deriving performance properties for each of the oils and/or surfactant and the further component from the identifier.

5. The method of claim 1, wherein the performance properties of each of the different oils and/or surfactant and the further component relate to physico-chemical properties of each of the different oils and/or surfactant and the further component.

6. The method of claim 1, wherein the performance properties of each of the different oils and/or surfactant and the further component relate to sensory properties of each of the different oils and/or surfactant and the further component.

7. The method according to claim 1, wherein oil-containing product for cosmetics comprises at least two different oils, at least three different oils, or at least four different oils.

8. The method of claim 1 further comprising the step of providing to the processing device via the communication interface target performance properties of a specific oil or a mixture of oils and/or surfactant and the further component or a mixture of surfactant and the further component for cosmetics.

9. The method of claim 8, wherein the method step of providing target performance properties is preceded by the steps
providing an identifier of a specific oil or a specific mixture of oils and/or a specific surfactant or a specific further component or a specific mixture of surfactant and the further component,
deriving the target performance of the specific oil or the specific mixture of oils properties and/or of the specific surfactant or the specific further component or the specific mixture of surfactant and the further component from the identifier of the specific oil or the specific mixture of oils and/or of the specific surfactant or the specific further component or the specific mixture of surfactant and the further component.

10. The method according to claim 8, wherein the method further comprises the step of comparing by the processing device the target performance properties of a specific oil or a mixture of oils and/or a specific surfactant or a specific further component or a specific mixture of surfactant and the further component for cosmetics with the determined performance properties of the oil-containing and/or the surfactant-containing product for cosmetics and deriving a result of the comparing step, wherein the step of comparing comprises comparing if the target performance requirements are met, wherein providing via the output channel further comprises providing the result of the comparing step.

11. The method of claim 10, further comprising the step of varying the composition parameters, further comprising the step of providing the varied composition parameters as composition parameters.

12. The method of claim 11, further comprising the step of repeating the method, until the target performance properties are met.

13. The method of claim 11, wherein the step of varying the composition parameter comprises varying the measure for the ratio of the different oils and/or the surfactant or the further component and/or, wherein step of varying the composition parameter comprises changing at least one identifier of the different oils and/or the surfactant or the further component and or comprises adding an additional identifier of an oil and/or of a surfactant or of a further component.

14. The method of claim 8, wherein the target performance properties of a specific oil or a mixture of oils for cosmetics relate to a silicone-based oil or a mineral/paraffin oil.

15. The method of claim 8, wherein the target performance properties of a specific surfactant or a specific further component or a mixture of surfactant and further component relate to alkoxylate and sulfate based surfactants.

16. A system (12) for determining performance properties of an oil-containing and/or surfactant-containing product for cosmetics, the oil-containing product for cosmetics comprising different oils forming a mixture and/or the surfactant-containing product for cosmetics comprises at least one surfactant and further component forming the mixture, the system (12) comprising:
a communication interface (18), and
a processing device (16) configured to perform the method according to claim 1.

17. A computer program product that, when run on a processing device performs the method according to claim 1.

18. A method for determining performance properties of an oil-containing and/or a surfactant-containing product for cosmetics comprising the use of a system according to claim 16 for production of an oil containing product and/or of a surfactant-containing product for cosmetics.
